(19) United States
(12) Patent Application Publication
(10) Pub. No.: US 2006/0024712 A1
(43) Pub. Date: Feb. 2, 2006

(54) Separation of nucleic acid

(75) Inventors: Matthew J. Baker, Maidstone (GB); Anthony Stevenson, Iwade (GB); John Buckels, Canterbury (GB)

Correspondence Address: VINSON & ELKINS, L.L.P., 1001 FANNIN STREET, 2300 FIRST CITY TOWER, HOUSTON, TX, 77002-6760, US

(73) Assignee: INVITROGEN CORPORATION

(21) Appl. No.: 11/167,907

(22) Filed: Jun. 27, 2005

Related U.S. Application Data
(60) Provisional application No. 60/582,879, filed on Jun. 25, 2004.

(30) Foreign Application Priority Data
Oct. 7, 2004 (GB) 0422299.8
Jun. 25, 2004 (GB) 0414302.0

(51) Int. Cl. C12Q 1/68; C12N 1/08

(52) U.S. Cl. 435/6; 435/270

(57) ABSTRACT

Compositions, methods and kits for separating nucleic acid from cell samples. Cells are lysed and nuclear material is flocculated/precipitated. Genomic DNA can be collected from the precipitate and purified. RNA present in the supernatant can be collected (e.g., bound to a solid phase) and purified.

### CROSS-REFERENCE TO RELATED APPLICATIONS

This application claims priority to Great Britain Patent Application Number 04143020, filed Jun. 25, 2004, and 04222998, filed Oct. 7, 2004.

### FIELD OF THE INVENTION

The present invention provides compositions and methods for separating genomic DNA and RNA from other cellular components.

### BACKGROUND OF THE INVENTION

Separating genomic DNA and RNA from other components of the cell is a challenging problem, and one that has not yet been solved in a simple way that is amenable to automation and that avoids the use of undesirable reagents or conditions. Many existing approaches for isolating nucleic acid are labor intensive, use toxic or hazardous reagents, and/or can damage the resulting nucleic acid. The present invention provides a straightforward method for isolating genomic DNA and RNA from cells.

### SUMMARY OF THE INVENTION

One embodiment of the present invention is a method for separating genomic DNA from a cell sample, comprising lysing the cells under conditions which do not employ strongly chaotropic or denaturing conditions or reagents (“non-denaturing conditions”), to flocculate the genomic DNA and form a precipitate; and collecting the precipitate. In one aspect, the cells are lysed with a hypertonic monovalent cationic salt solution at a pH between about 2.0 and 12.0, 4.0 and 10.0, 6.0 and 9.0 or 6.0 and 8.0. The monovalent cationic salt may be an alkali metal cation salt or an ammonium salt. In one embodiment, the concentration of the salt solution is between about 10 mM and 1.0 M. In another embodiment, the salt is sodium chloride (NaCl), potassium chloride (KCl), sodium carbonate (Na₂CO₃), sodium bicarbonate (NaHCO₃), ammonium bicarbonate (NH₄HCO₃), lithium chloride (LiCl) or Cesium Chloride (CsCl). In one aspect, the pH of the salt solution is between about 4.0 and 10.0. The salt solution may further comprise about 0.1 to 1.0% v/v of a non-ionic detergent.

In another embodiment, the cell lysis occurs in the presence of a solid phase capable of binding the genomic DNA. The solid phase may bind at least about 50·g, 60·g, 70·g, 80·g, 90·g or 100·g of genomic DNA per mg of solid phase. The method may further comprise contacting the solid phase with a solution under conditions to release the precipitate of the genomic DNA and nuclear material into the solution; treating the solution to remove one or more impurities; and rebinding the genomic DNA to the solid phase. The treating step may further comprise contacting the solution with a protease. In one embodiment, the rebinding step further comprises adding a precipitant, whereby the genomic DNA rebinds to the solid phase. In another embodiment, the solid phase is a charge switch material. The solid phase may be a spooling rod, a bead or particulate composition, a single bead, a mesh, a membrane, a sinter, a plastic support, a paper, a tip, a dipstick, a wall of a container, a tube, a well, a probe, a pipette, a filter, a sheet, a slide or a plug.

In another aspect, the lysing does not involve using one or more of:

(i) a chaotropic reagent,

(ii) a strong ionic detergent;

(iii) a pH that is above about 10.0, 11.0 or 12.0 or below about 4.0, 3.0 or 2.0;

(iv) a divalent or trivalent metal ion; or

(v) a protein precipitant.

In yet another embodiment, the method does not involve ultracentrifugation. In yet another embodiment, at least about 50% (e.g, at least about 60%, 70%, 80% or 90%) of the protein initially present in the cell sample is removed. In one embodiment, the cell sample is a mammalian cell sample or a blood cell sample. The cell sample may be a whole blood cell sample. In one embodiment, the precipitate, or a nucleic acid sequence within the genomic DNA, is amplified.

The present invention also provides a kit for separating genomic DNA from a cell sample, comprising a volume of a hypertonic solution of a monovalent cation salt having a pH between about 2.0 and 12.0, 4.0 and 10.0, 6.0 and 9.0 or 6.0 and 8.0, for lysing the cells and flocculating the genomic DNA and nuclear material. The kit may comprise 0.1 to 1.0% of a non-ionic detergent. The kit may also comprise instructions for enriching the nuclear material and genomic DNA. In one embodiment, the kit further comprises a solid phase for binding the precipitated DNA. In another embodiment, the kit further comprises an elution reagent for releasing the DNA from the solid phase. The kit may further comprise one or more of a protease chaotropic reagent or denaturant. In one aspect, the kit further comprises an alcohol and/or a soluble charge switch material for rebinding DNA onto the solid phase.

The present invention also provides a method for separating RNA from a cell sample, comprising lysing the cells under non-denaturing conditions to flocculate genomic DNA and to form a precipitate and a supernatant; and collecting the supernatant from the precipitate, wherein the supernatant contains the RNA. In one embodiment, the cells are lysed with a hypertonic monovalent cationic salt solution at a pH between about 2.0 and 12.0, 4.0 and 10.0, 6.0 and 9.0 or 6.0 and 8.0. The monovalent cationic salt may be an alkali metal cation salt or an ammonium salt. In one embodiment, the concentration of the salt solution is between about 10 mM and 1.0 M. In another embodiment, the salt is sodium chloride (NaCl), potassium chloride (KCl), sodium carbonate (Na₂CO₃), sodium bicarbonate (NaHCO₃), ammonium bicarbonate (NH₄HCO₃), lithium chloride (LiCl) or Cesium Chloride (CsCl). In one aspect, the pH of the salt solution is between about 4.0 and 10.0. The salt solution may further comprise about 0.1 to 1.0% v/v of a non-ionic detergent.

In another embodiment, the cell lysis occurs in the presence of a solid phase capable of binding the genomic DNA. The solid phase may bind at least about 50·g, 60·g, 70·g, 80·g, 90·g or 100·g of genomic DNA per mg of solid phase. The method may further comprise: contacting the solid phase with a solution under conditions to release the precipitate of the genomic DNA and nuclear material into the solution; treating the solution to remove one or more impurities; and rebinding the genomic DNA to the solid phase. The treating step may further comprise contacting the solution with a protease. In one embodiment, the rebinding step further comprises adding a precipitant, whereby the genomic DNA rebinds to the solid phase. In another embodiment, the solid phase is a charge switch material. The solid phase may be a spooling rod, a bead or particulate composition, a single bead, a mesh, a membrane, a sinter, a plastic support, a paper, a tip, a dipstick, a wall of a container, a tube, a well, a probe, a pipette, a filter, a sheet, a slide or a plug.

In another aspect, the lysing does not involve using one or more of:

(i) a chaotropic reagent;

(ii) a strong ionic detergent;

(iii) a pH that above about 10.0, 11.0 or 12.0 or below about 4.0, 3.0 or 2.0;

(iv) a divalent or trivalent metal ion; or

(v) a protein precipitant.

In another embodiment, the method does not involve ultracentrifugation. In yet another embodiment, at least about 50% (e.g, at least about 60%, 70%, 80% or 90%) of the protein initially present in the cell sample is removed. In one embodiment, the cell sample is a mammalian cell sample or a blood cell sample. The cell sample may be a whole blood cell sample. The method may further comprise purifying the RNA.

### DETAILED DESCRIPTION OF THE INVENTION

The present invention provides compositions, methods and kits for separating nucleic acid, particularly genomic DNA and RNA, from cells. Cells are chemically lysed, resulting in flocculation of nuclear material including genomic DNA, nuclear proteins and other nuclear components. Cells may be lysed in the presence of a solid phase optionally coated with a “charge switch material” as described in detail herein, or the solid phase may be added after the lysis step. Genomic DNA is then collected from the flocculated nuclear material (referred to herein as the “precipitate”) and purified. RNA present in the supernatant can be collected (e.g., bound to a solid phase) and purified. The disclosed methods are useful for separating genomic DNA from other cellular components (e.g., non-genomic DNA, soluble RNA (e.g., mRNA), proteins, polypeptides and other cytoplasmic components) remaining in the supernatant, and enable the genomic DNA to be further manipulated or analyzed.

The methods described herein may be used to isolate genomic nucleic acid, such as host cell chromosomes, genomic DNA, ribosomal RNA, and mitochondrial DNA, thereby enabling the separation of target genomic nucleic acid from non-genomic nucleic acid. Non-genomic nucleic acid generally has a much lower molecular weight than genomic DNA and tends not to flocculate when contacted with the reagents described herein, and includes vectors, plasmids, self replicating satellite nucleic acid or cosmid DNA, vector RNA, bacteriophages (e.g. phage lambda and M13) and viral nucleic acids.

Cell Lysis and Flocculation of Nuclear Material

In the present methods, cells are lysed under conditions which do not employ strongly chaotropic or denaturing reagents. By “strongly chaotropic”, it is meant that the concentrations of chaotropic agent (if present) do not result in substantial protein denaturation. In one embodiment, a chaotropic reagent is present at a concentration of less than about 2M, less that about 1.5M, less than about 1M, less that about 500 mM or less than about 100 mM. It will be appreciated that the concentration at which a particular chaotrope acts as a denaturant will vary, and is either well known in the art or may be determined using well known methods.

Both the cell and nuclear membranes are lysed using the methods described herein. A solid phase optionally coated with a “charge switch material” is either present during the lysis step, or is added after the lysis step, resulting in the binding of flocculated nuclear material. Genomic DNA contained within the nuclear material is thus flocculated and separated from other components of the cell. The solid phase facilitates separation or further processing of the flocculated nuclear material. Other methods for separating nuclear material from cell lysates include settling under gravity, filtration, electrochemical techniques, dialysis, ultrafiltration trapping the nuclear precipitate in the small channels of a microfluidic circuit. In one embodiment, the separation of flocculated nuclear material does not involve centrifugation, a technique commonly used in conjunction with density gradients for the separation of cell nuclei from the supernatants obtained after cell lysis and the removal of cell debris.

Surprisingly, the present invention allows straightforward isolation of genomic DNA using non-toxic reagents that do not substantially compromise the integrity of the resulting genomic DNA for subsequent manipulation or analysis. The flocculation of genomic DNA also results in the enrichment of RNA which is present in the supernatant resulting from flocculation of genomic DNA and nuclear material. Once the genomic DNA precipitate is obtained, the other components present in the supernatant (e.g. RNA, particularly mRNA) may be isolated by standard methods as described below. The resulting RNA is also substantially intact and may be further manipulated or analyzed.

Reagents and conditions for cell lysis include monovalent cationic salts, particularly hypertonic solutions of these salts. Examples of suitable monovalent salts include alkali metal cationic salts (e.g., lithium, sodium, potassium) or ammonium salts. The counter-ion may be a halide, carbonate, or bicarbonate ion. Examples of suitable salts include sodium chloride (NaCl), potassium chloride (KCl), sodium carbonate (Na₂CO₃), sodium bicarbonate (NaHCO₃) and ammonium bicarbonate (NH₄HCO₃). The concentration of the salt solutions employed in the present invention is generally between about 5 mM and 2.0 M, and may also be between about 10 mM and 1.0 M. Specific embodiments utilize sodium chloride (NaCl) solutions between about 0.25 and 1.0 M or ammonium bicarbonate (NH₄HCO₃) between about 0.5 and 1.0 M.

In one embodiment, pH conditions between about 4.0 and 10.0 are used to lyse the cells. In other embodiments, pH conditions between about 6.0 and 9.0, or between about 7.0 and 9.0 are used.

In another embodiment, a non-ionic detergent is included in the lysis buffer. (e.g., Tween 20, Triton X-100 or Nonidet P-40). The non-ionic detergent can be used, for example, as an about 0.1 to 1.0% v/v solution.

In other aspects, the lysis step does not involve using one or more of:

(i) chaotropic reagents, such as high concentrations of guanidine or urea; and/or

(ii) strong ionic detergents such as sodium dodecyl sulfate (SDS) or lauryl sarcosine; and/or

(iii) a pH above about 10.0 or below about 4.0, in particular avoiding the use of strong mineral bases such as NaOH or a strong mineral acids such as HCl; and/or

(iv) divalent or trivalent metal ions such as Mg²; and/or

(v) reagents that cause gross protein precipitation, such as known protein precipitants for example, polyethylene glycols, alcohols, miscible organic solvents or certain salts known in the art, such as sulfates and phosphates.

These reagents are commonly used to purify DNA, but generally have the disadvantage of contaminating the DNA containing sample, for example by causing the co-precipitation of substantial amounts of protein in the sample, or degrading the target DNA. In one embodiment, the steps of the method above do not use any of the reagents or conditions (i)-(v). However, the method may avoid the use of one, any two, any three or any four of the conditions, and the avoidance of all combinations and permutations of these conditions is within the scope of the present embodiments. In other embodiments, low levels of protein precipitants (e.g., less than about 5%, may be used.

Examples of solid phases include a spooling rod, beads or particulates, single beads, a mesh, a membrane, a sinter, a plastic support, a paper, a tip, a dipstick, a wall of a container, a tube, a well, a probe, a pipette, a filter, a sheet, a slide or a plug, any of which could possess ionizable groups. Since the precipitate of nuclear material containing DNA is very sticky, it can be adhered to a wide range of solid supports used to separate it from the cell lysate. The solid phase may be particulate (e.g., a bead). The solid phase may also be magnetizable to aid in the separation and manipulation of the solid phase. One solid phase is a magnetic bead. The solid phase can be formed from glass, silica, plastic, a mineral, a carbohydrate, paper, or a natural product such as cellulose, and combinations thereof. The present methods may employ particularly small amounts of solid phase compared to the initial sample volume which may be due to the level of enrichment provided in the initial steps of the method.

The ability of the methods described herein to use comparatively small amounts of solid phase provide the further feature that in the subsequent processing of the DNA bound to the solid phase, small elution volumes can be used to release the DNA from the solid phase. For example, the method may employ about 3 mg of magnetic beads to bind about 400·g of DNA. This amount of DNA and beads can easily be eluted into a volume of 1 ml, with the volume of the beads contributing negligibly to the overall sample volume (e.g., 3 mg of bead occupies a volume of about 10·l). The ability to use small amounts of solid phase also allows the DNA bound to the beads to be further treated, e.g. releasing the sample from the solid phase and contacting it with a digestion buffer to remove trace proteins and then rebinding the DNA to the solid phase, for example by using a precipitant such as an alcohol, polyethylene glycol or a soluble charge switch material such as Poly Bis-Tris to help redeposit the DNA on the solid phase.

In one embodiment, the flocculated nuclear material (e.g., genomic DNA) is separated from cell walls and/or proteins and/or lipids and/or carbohydrates released from the lysed cells. The present methods provide enrichment factors of the genomic DNA present in the sample compared to the total protein of at least about 4 times, at least about 10 times, at least about 20 times, or at least about 50 times. This feature is useful, for example, in the processing of whole blood samples as a 10 ml sample of blood contains at least 1 gram of protein. The processing of such samples according to the methods disclosed herein provides pellets of nuclear material containing the target DNA having less than about 50mg, 40 mg, 30 mg, 20mg or even 10 mg of protein, thereby representing enrichment factors of the protein in the sample of about 20:1, 25:1, 33.3:1, 50:1 or 100:1. While other types of eukaryotic cells generally contain less protein than blood samples, the present invention generally enables at least about 50%, 60%, 65%, 70%, 75%, 80%, 85%, 90% or 95% of the protein initially present in the cell sample to be removed using this method.

The method may comprise binding the precipitate and the further component, particular an RNA component, to a single solid phase which is capable of differential release of the precipitate and the further component. For example, the solid phase may be a charge switch solid phase which is capable of releasing the RNA (but not the precipitated DNA) when the pH is adjusted (e.g., from low to high pH), as described in more detail below. The solid phase with bound precipitate and RNA can be isolated from the solution containing the further contaminant, and the RNA eluted in fresh solution.

Hence, in some embodiments of the invention, the method may comprise binding RNA and/or DNA to a solid phase, which may be the same (i.e., a single solid phase to which the RNA and DNA are bound) or different. Reagents as described above may be used to remove any residual protein from the fresh solution.

Cells suitable for use in the compositions and methods described herein include eukaryotic cells, for example, fungal cells (e.g., yeast cells), animal cells (e.g., cultured cells) and plant cells. Plant cells may be treated with a cell wall-degrading enzyme such as cellulase prior to processing using the methods described herein. Animal cells may be from a mammalian (e.g., human) tissue or organ, such as the liver, kidney, pancreas, heart, spleen, lung, skin, stomach, intestine, prostate, brain, muscle, breast, prostate or any other tissue or organ type. In addition, blood cells (e.g. whole blood) may be used.

The methods disclosed herein are particularly useful for larger scale nucleic acid (e.g. genomic DNA) purifications, for example those involving initial sample volumes which are at least about 15 ·l and more preferably at least about 300·l, for example containing at least about 100,000 cells and more preferably at least about 200,000 cells. These preparations generally aim to provide at least about 1·g and more preferably at least about 2·g of gDNA.

Collection of Genomic DNA

After the precipitate has been bound to a solid phase, it may be desirable to remove residual protein forming part of the precipitate to obtain a substantially pure DNA sample. Such samples may be, for example, 50%, 60%, 70%, 80%, 90%, 95% or 99% free of contaminating proteins. This can be done using means well known in the art, for example by heating (e.g. in a PCR reaction) or by shearing forces (e.g., vortexing or shearing with a pipette) or contacting with one or more proteases, denaturants or chaotropes. Chaotropes and denaturants digest the precipitate, followed by the use of a DNA precipitant such as an alcohol. These steps may help to concentrate the DNA onto a small amount of solid phase. However, other techniques for separating the DNA from residual protein contaminants include the use of charge switch material, anion exchangers or any one of a range of other methods known to those skilled in the art. Examples of purification techniques include ion-exchange, electrophoresis, silica solid phase with chaotropic salt extraction, precipitation, dialysis, flocculation, ultra filtration, filtration, gel filtration, centrifugation, alcohol precipitation and/or the use of a charge switch material as described below.

In other aspects, the precipitate may be treated according to other well-known methods for the purification of DNA from other materials in the precipitate. For example, the step of purifying the DNA may comprise contacting the precipitate with an ionic detergent such as SDS and proteinase K for degrading and removing contaminating protein.

Collection of RNA

In this embodiment, the precipitate is discarded and the RNA contained in the supernatant is isolated by conventional methods including ethanol precipitation, column chromatography and charge switch magnetic beads. The RNA may then be bound to a solid surface, including a microwell plate, tube or other container which is coated with a charge switch material. In any of the genomic DNA separation methods described herein, the supernatant resulting from the DNA flocculation step may be used as a source of RNA. In this method, at least about 75%, at least about 80%, at least about 90%, at least about 95%, at least about 99% or at least 99.9% of the genomic.DNA is removed. Such RNA samples may be, for example, 50%, 60%, 70%, 80%, 90%, 95% or 99% free of contaminating proteins.

In one embodiment, chaotropic reagents, strong ionic detergents and/or agents which cause precipitation of RNA such as alcohol or polyethylene glycol are used to separate RNA from protein. Hence, as described above, the step of removing the DNA may be carried out without employing strongly chaotropic or denaturing conditions or reagents, but once the DNA has been removed chaotropic agents and the like can be used for further purification steps. For example, chaotropic reagents may be used to allow the proteins to remain in solution while the RNA is bound to a solid phase or otherwise separated. Where the solid phase is a charge switch solid phase, then urea is a suitable chaotrope.

Kits

In a further aspect, kits are provided for carrying out the methods disclosed herein. By way of example, the kits for separating nuclear material containing DNA from a sample of cells, e.g., enriching nuclear material containing target DNA present in a sample of cells, may comprise:

(a) a volume of a hypertonic solution of a monovalent cation salt having a pH between 6.0 and 9.0, and optionally comprising 0.1 to 1.0% of a non-ionic detergent, for lysing the cells; and/or

(b) instructions for using the kit to separate the flocculated nuclear material resulting from cell lysis, or to enrich the nuclear material and DNA present in the cells; and/or

(c) an amount of solid phase for binding the flocculated DNA; and/or

(d) a volume of an elution reagent for releasing the DNA from the solid phase; and/or

(e) a volume of a digestion reagent comprising one or more proteases and/or one or more chaotropic reagents and/or one or more denaturants for use in removing residual protein after the initial flocculation reaction;

(f) a volume of an alcohol (e.g. propanol) and/or a soluble charge switch material for rebinding DNA onto the solid phase, e.g. after a releasing step and treatment to remove one or more impurities such as residual protein. Preferably such as reagent is a buffer having a pH of about 4.0 to 6.0.

In a further aspect, the method may provide kits for separating DNA (or RNA) from a sample including DNA and at least one further component (e.g., RNA), to provide a solution containing the further component(s), which kit may comprise:

(a) a volume of a hypertonic solution of a monovalent cation salt having a pH between 6.0 and 9.0, and optionally comprising 0.1 to 1.0% of a non-ionic detergent, for lysing the cells; and/or

(b) instructions for using the kit to separate the flocculated nuclear material, to provide a solution containing the further component(s); and/or

(c) an amount of solid phase for binding the further component and/or the flocculated DNA; and/or

(d) a volume of an elution reagent for releasing the further component from the solid phase; and/or

(e) a volume of a digestion reagent comprising one or more proteases and/or one or more chaotropic reagents and/or one or more denaturants for use in removing protein from the solution; and/or

(f) a volume of an alcohol (e.g. propanol) and/or a soluble charge switch material for rebinding the further component (e.g. RNA) onto the solid phase, e.g. after a releasing step and treatment to remove one or more impurities such as residual protein. The reagent may be a buffer having a pH of about 4.0 to 6.0.

Other preferred components or features of the kits are as described above in relation to the methods. Embodiments of the present invention will now be described in more detail by way of example and not limitation.

Amplification of Isolated Nucleic Acid

The present methods may also comprise the further step of amplifying nucleic acid from the flocculated nuclear material, with or without additional purification. This is possible since the initial treatment of the cells does not employ conditions which are incompatible with the PCR reaction and is capable of providing DNA sample sizes and levels of enrichment which are compatible with direct amplification of target sequences present in the DNA sample.

The target nucleic acid may be conveniently amplified using PCR (or RT-PCR). These techniques are described, for example, in U.S. Pat. No. 4,683,195. In general, such techniques require that sequence information from the ends of the target sequence is known to allow suitable forward and reverse oligonucleotide primers to be designed to be identical or similar to the polynucleotide sequence that is the target for the amplification. PCR comprises the steps of denaturation of template nucleic acid (if double-stranded), annealing of primer to target, and polymerization. The nucleic acid probed or used as the template in the amplification reaction may be genomic DNA, cDNA or RNA. PCR can be used to amplify specific sequences from genomic DNA, specific RNA sequences and cDNA transcribed from mRNA, bacteriophage or plasmid sequences. The general use of PCR techniques is described in Mullis et al, Cold Spring Harbor Symp. Quant. Biol., 51:263, (1987), Ehrlich (ed), PCR Technology, Stockton Press, NY, 1989, Ehrlich et al, Science, 252:1643-1650, (1991), “PCR protocols; A Guide to Methods and Applications”, Eds. Innis et al, Academic Press, New York, (1990).

Charge Switch Materials

Charge switch materials are described in PCT WO 99/29703 and PCT WO 02/48164, the entire contents of which are incorporated herein by reference, and many of these materials, in particular the water soluble polymers and biological buffers, can be used in accordance with the compositions and methods described herein. Charge switch materials can be used for binding nucleic acid present in a sample by contacting the sample with the charge switch material at a first pH at which the charge switch material has a positive charge and will bind negatively charged nucleic acid, and then releasing the nucleic acid at a second, higher pH at which the charge switch material possesses a neutral, negative or less positive charge than at the first. pH. In alternative embodiments, charge switch materials can also be used to bind positively charged target substances, in this case binding them at a first pH and then releasing the substances at a second, lower pH at which the charge switch material is neutral, positive or less negative than the first pH.

Generally the charge switch material will possess an overall positive charge, that is the sum of all positive and negative charges on the charge switch material as a whole is positive. It is possible, however, that the charge switch material as a whole could be negatively charged, but have areas of predominantly positive charge to which the nucleic acid can bind. The change in the charge of the material is referred to herein as “charge switching” and is accomplished by the use of a “charge switch material”.

The charge switch material comprises an ionizable group which changes charge according to the ambient conditions. The charge switch material is chosen so that the pKa of the ionizable group is appropriate to the conditions at which it is desired to bind nucleic acid to and release nucleic acid from the charge switch material. Generally, nucleic acid will be bound to the charge switch material at a pH below or about equal to the pKa, when the charge switch material is positively charged, and will be released at a higher pH (usually above the pKa), when the charge switch material is less positively charged, neutral, or negatively charged.

Similarly, in referring to positively and negatively charged target substances, it is generally meant that the net overall charge of the target substance is positive or negative In some circumstances, a target substance may have charged regions of an opposite charge to the overall net charge that can be bound by an appropriate reagent.

In other embodiments, charge switch materials allow binding and/or releasing (especially releasing) of the nucleic acid to occur under mild conditions of temperature and/or pH and/or ionic strength.

Generally, the charge switch material will change charge because of a change in charge on a positively ionizable group from positive to less positive or neutral, as the pH is increased in a range spanning or close to the pKa of the positively ionizable group. This may also be combined with a change in charge on a negatively ionizable group from neutral or less negative to more negative.

The charge switch material may comprise an ionizable group having a pKa between about 3 and 9. For positively ionizable groups, the pKa is at least about 4.5, 5.0, 5.5, 6.0 or 6.5 and/or at most about 8.5, 8.0, 7.5 or 7.0. In some embodiments, the pKa of the positively ionizable group is between about 5 and 8; between about 6.0 and 7.0, or between about 6.5 and 7.0. The pKa for negatively ionizable groups may be between about 3.0 and 7.0, or between about 4.0 and 6.0, which is the approximate pH at which nucleic acid is bound.

Materials having more than one pKa value (e.g. having different ionizable groups), or combinations of materials having different pKa values, may also be suitable for use as charge switch materials, provided that at a first (lower) pH the material(s) possess(es) a positive charge and that at a higher pH the charge is less positive, neutral or negative.

Generally, a charge switch is achieved by changing the pH from a value below to a value above the pKa of the ionizable group. However, it will be appreciated that when the pH is the same as the pKa value of a particular ionizable group, 50% of the individual ionizable groups will be charged and 50% will be neutral. Therefore, charge switch effects can also be achieved by changing the pH in a range close to, but not spanning, the pKa of an ionizable group. For example, at the pKa of a negatively ionizable group, such as a carboxy group (pKa about 4), 50% of such groups will be in the ionized form (e.g., COO⁻) and 50% will be in the neutral form (e.g. COOH). As the pH increases, an increasing proportion of the groups will be in the negative form.

In one embodiment, the binding step is carried out at a pH of below the pKa of the ionizable group, or within about 1 pH unit above the pKa. Generally, the releasing step is carried out at a pH above the pKa of the ionizable group (e.g., at a pH between 1 and 3 pH units above the pKa).

The use of strong bases, or weak bases in combination with heating, as described in EP 0 707 077 A, can also lead to degradation of RNA (especially at pH values of 10 or above), and denaturation of double stranded DNA (i.e. irreversible conversion of DNA from the double stranded form at least partially into the single stranded form), which can lead to a lack of specific binding in PCR.

The appropriate choice of pKa value(s) as described herein allows the step of releasing nucleic acid from the solid phase to be performed under mild conditions. As used herein, the term “mild conditions” generally means conditions under which nucleic acid is not denatured and/or not degraded and/or not depurinated, and/or conditions which are substantially physiological.

The releasing step may be performed at a pH of no greater than about pH 10.5, 10.0, 9.8, 9.6, 9.4, 9.2, 9.0, 8.9, 8.8, 8.7, 8.6 or 8.5. Depending on the pKa(s) of the charge switch material, the releasing step may even be performed at lower pH values, such as 8.0, 7.5 or 7.0. In one embodiment, the releasing step is carried out in the substantial absence of NaOH, and/or in the substantial absence of other alkali metal hydroxides, and/or in the substantial absence of strong mineral bases. Substantial less than 20 mM, less than 15 mM or less than 10 mM.

The desired change in pH can be achieved by altering the ionic strength of the solution and/or the temperature, since pH is dependent on both these factors. However, neither high temperature nor high ionic strength are generally compatible with the desired mild conditions, and accordingly, the change in pH is generally not achieved by large changes in ionic strength or temperature. Moreover, increasing ionic strength increases competition of charged species with the nucleic acid for binding to the charge switch material, which may assist in releasing the nucleic acid. Small changes of ionic strength are therefore acceptable and may be used in conjunction with the change in pH to release the nucleic acid, (e.g., within the limits and ranges given below).

The temperature at which the releasing step is performed is generally no greater than about 70° C., 65° C., 60° C., 55° C., 50° C., 45° C. or 40° C. Such temperatures may also apply to the entire process. The releasing step, or the entire process, may even be performed at lower temperatures, such as 35° C., 30° C. or 25° C.

Furthermore, the releasing step may occur under conditions of low ionic strength, (e.g., less than 1M, 500 mM, 400 mM, 300 mM, 200 mM, 100 mM, 75 mM, 50 mM, 40 mM, 30 mM, 25 mM, 20 mM, 15 mM, 10 mM. The ionic strength may be at least about 5 mM, or at least about 10 mM. These ionic strengths may also apply to the binding step.

PCR is sensitive to pH and the presence of charged contaminants. In certain embodiments, the releasing step is performed using reagents suitable for storing nucleic acid (such as a commercially available storage buffer, e.g. 10 mM Tris-HCl, pH8.0-8.5, optionally in the presence of 1 mM EDTA), or using reagents suitable for use in a procedure to which the nucleic acid is to be subjected (such as a PCR buffer, e.g. 10 mM Tris-HCl, 50 mM KCl, pH 8.5).

Conventional nucleic acid extraction processes often require a step of diluting the elution product containing nucleic acid to make the solution suitable for PCR. In one embodiment, the present methods substantially avoid diluting the released nucleic acid.

In one embodiment, the step of binding DNA occurs under mild conditions, (e.g., at a pH of no less than about 3.0, an 3.5, 4.0, 4.5 or 5.0). Previous methods have used high concentrations of chaotropic agents, such as 8M guanidine. Such conditions may not be necessary in the practice of the present invention, in which the binding step may occur in solution having a total concentration of 1M or less. Other temperatures and ionic strengths are as detailed above for the releasing step.

The use of such mild conditions for the release of nucleic acid is especially useful for extracting small quantities of nucleic acid, as the extracted DNA or RNA can be added directly to a reaction or storage tube without further purification steps, and without the need to dilute high ionic strength. Therefore, loss of nucleic acid through changing the container, imperfect recovery during purification steps, degradation, or denaturation, and dilution of small amounts of nucleic acid can be avoided. This is particularly advantageous when a nucleic acid of interest is present in a sample (or is expected to be present) at a low copy number, such as in certain detection and/or amplification methods.

Chemical species for use as charge switch materials in accordance with the invention comprise a positively ionizable nitrogen atom, and at least one electronegative group (such as a hydroxy, carboxy, carbonyl, phosphate or sulfonic acid group) or double bond (e.g. C=C double bond), which is sufficiently close to the nitrogen atom to lower its pKa. It has been found that such molecules tend to have suitable pKa values for the extraction of nucleic acid under mild conditions according to the present invention. In one embodiment, at least one electronegative group is separated from the ionizable nitrogen by no more than two atoms (usually carbon atoms).

In one embodiment, hydroxyl groups are the electronegative groups used (particularly when several hydroxyl groups are present, e.g. in polyhydroxyl amines, such as Tris (C(CH₂OH)₃—NH₂) or Bis-Tris (see below)), as they (1) lower the pKa of the nitrogen atom (e.g. amine group, e.g. from about 10 or 11) to a suitable value around neutral (i.e. pKa of about 7), (2) allow the species to remain soluble/hydrophilic above the pKa, when the nitrogen atom of the amine group loses its positive charge, (3) provide a site for covalent linkage to a tagging groups and/or solid substrates, e.g. a polycarboxylated polymer (such as polyacrylic acid), and (4) are uncharged at pH values suitable for the releasing step and at which procedures such as PCR are performed (typically pH 8.5); the presence of charged species can interfere with PCR especially. Other suitable chemical species have an ionizable nitrogen atom and at least 2, 3, 4, 5 or 6 hydroxyl groups. Further examples of polyhydroxylated amines are dialcohol amine reagents such as diethanol amine. In one embodiment, silane reagents based on these compounds can be used to attach [HO—(CH₂)ₙ]₂—N—(CH₂)ₘ— moieties, where n and m are selected from 1 to 10, to tagging groups.

Many standard, weakly basic, buffers also contain suitable chemical species to provide the ionizable groups of charge switch materials, as they have pKa values close to neutral (i.e. 7).

The charge switch reagents may also be derivatized so that they are linked to a member of a specific binding pair and used in accordance with the disclosure in, for example, PCT/GB2003/005496.

Solid phases that can be derivatized with charge switch materials include beads, particles, tubes, wells, probes, dipsticks, pipette tips, slides, fibers, membranes, papers, celluloses, agaroses, glass or plastics) in a monomeric or polymeric form via adsorption, ionic or covalent interactions, or by covalent attachment of the binding partner to a polymer backbone which is in turn immobilized onto the solid support.

Solid phase materials, especially beads and particles, may be magnetizable, magnetic or paramagnetic. This can aid removal of the solid phase from a solution containing the released nucleic acid, prior to further processing or storage of the nucleic acid.

In one embodiment, the weakly basic buffers are biological buffers, i.e. buffers from the class of buffers commonly used in biological buffer solutions such as HEPES, PIPES, MOPS, and many others which are available from suppliers such as Sigma (St. Louis, Mo.).

Leaching (i.e. transfer from the solid phase into solution in the liquid phase) of chemical species used to provide ionizable groups in ion exchange resins occurs to some extent, especially when the species are immobilized on the solid phase by the interaction of the specific binding pair. Such leaching typically causes impurities in the resultant product, which can lead to significant problems, particularly if the resultant product is intended to be used in PCR (and especially when the species are charged). The use of biological buffers to provide the ionizable groups in charge switch materials avoids this problem, since leaching of such buffers into the liquid phase will generally not significantly affect the nucleic acid, nor any downstream processes such as PCR to which it might be subjected. Indeed, many biological buffers are routinely used in PCR buffers, storage buffers and other buffer solutions.

In one embodiment, the releasing step takes place in a buffer solution containing the same biological buffer that is used in, as or on the charge switch material portion of the reagent.

Examples of suitable biological buffers for use in charge switch materials, and their pKa values, are as follows:
N-2-acetamido-2-aminoethanesulfonic acid (ACES), pKa 6.8;
N-2-acetamido-2-iminodiacetic acid (ADA), pKa 6.6;
amino methyl propanediol (AMP), pKa 8.8;
3-1,1-dimethyl-2-hydroxyethylamino-2-hydroxy propanesulfonic acid (AMPSO), pKa 9.0;
N,N-bis2-hydroxyethyl-2-aminoethanesulfonic acid †\ (BES), pKa 7.1;
N,N-bis-2-hydroxyethylglycine (BICINE), pKa 8.3;
bis-2-hydroxyethyliminotrishydroxymethylmethane (Bis-Tris), pKa 6.5;
1,3-bistrishydroxymethylmethylaminopropane (BIS-TRIS Propane), pKa 6.8;
4-cyclohexylamino-1-butane sulfonic acid (CABS), pKa 10.7;
3-cyclohexylamino-1-propane sulfonic acid (CAPS), pKa 10.4;
3-cyclohexylamino-2-hydroxy-1-propane sulfonic acid (CAPSO), pKa 9.6;
2-N-cyclohexylaminoethanesulfonic acid (CHES) pKa 9.6;
3-N,N-bis-2-hydroxyethylamino-2-hydroxypropanesulfonic acid (DIPSO), pKa 7.6;
N-2-hydroxyethylpiperazine-N-3-propanesulfonic acid (EPPS or HEPPS), pKa 8.0;
N-2-hydroxyethylpiperazine-N-4-butanesulfonic acid (HEPBS), pKa 8.3;
N-2-hydroxyethylpiperazine-N-2-ethanesulfonic acid (HEPES), pKa 7.5;
N-2-hydroxyethylpiperazine-N-2-propanesulfonic acid (HEPPSO), pKa 7.8;
2-N-morpholinoethanesulfonic acid (MES), pKa 6.1;
4-N-morpholinobutanesulfonic acid (MOBS), pKa 7.6;
3-N-morpholinopropanesulfonic acid (MOPS), pKa 7.2;
3-N-morpholino-2-hydroxypropanesulfonic acid (MOPSO), pKa 6.9;
piperazine-N-N-bis-2-ethanesulfonic acid (PIPES), pKa 6.8;
piperazine-N-N-bis-2-hydroxypropanesulfonic acid (POPSO), pKa 7.8;
N-trishydroxymethyl-methyl-4-aminobutanesulfonic acid (TABS), pKA 8.9;
N-trishydroxymethyl-methyl-3-aminopropanesulfonic acid (TAPS), pKa 8.4;
3-N-trishydroxymethyl-methylamino-2-hydroxypropanesulfonic acid (TAPSO), pKa 7.4;
N-trishydroxymethyl-methyl-2-aminoethanesulfonic acid (TES), pKa 7.4;
N-trishydroxymethylmethylglycine (TRICINE), pKa 8.1; and trishydroxymethylaminomethane (TRIS), pKa 8.1;
histidine*, pKa 6.0, and polyhistidine;
imidazole*, pKa 6.9, and derivatives* thereof (i.e. imidazoles), especially derivatives containing hydroxyl groups**;
triethanolamine dimers**, oligomers** and polymers**; and di/tri/oligo amino acids**, for example Gly-Gly, pKa 8.2; and Ser-Ser, Gly-Gly-Gly, and Ser-Gly, the latter three having pKa values in the range 7-9.

In one embodiment, the buffers marked above with an asterisk (*) are not considered to be biological buffers in the compositions and methods described herein (whether or not they are designated as such in any chemical catalogue). In another embodiment, those marked with two asterisks (**) are also not considered to be biological buffers.

These and other chemical species comprising ionizable groups are typically employed as polymers, preferably following condensation polymerization.

Biological buffers and other chemical species comprising positively ionizable groups may be used in conjunction with a chemical species containing a negatively ionizable group which has a suitable pKa, for example in the ranges described above. For example, a biological buffer (having one or more positively ionizable nitrogen atoms) may be attached to a polymer or other solid phase material which has exposed carboxy groups even after attachment of the biological buffer. Such a material may bind nucleic acids at a low pH when few of the carboxy groups are negatively charged (i.e. few are in the COO⁻ form, most being in the COOH form) and most of the ionizable nitrogen atoms are positively charged. At higher pH the negative charge is stronger (i.e. a greater proportion of carboxy groups are in the COO⁻ form) and/or the positive charge is weaker, and the nucleic acid is repelled from the solid phase.

Chemical species containing ionizable groups (such as the biological buffers listed above) can be attached to a polymer backbone using known chemistries. For example a chemical species containing a hydroxyl group can be attached using carbodiimide chemistry to a carboxylated polymer backbones. Other chemistries can be employed by one of ordinary skill in the art using other polymer backbones (e.g. based on polyethylene glycol (PEG) or carbohydrate) using a range of standard coupling chemistries (see e.g. Immobilized Affinity Ligand Techniques, Greg T. Hermanson, A. Krishna Mallia and Paul K. Smith, Academic Press, Inc., San Diego, Calif., 1992, ISBN 0123423309, which is incorporated herein by reference in its entirety.)

Alternatively, the chemical species containing ionizable groups can be polymerized without a backbone polymer, using cross-linking reagents, for example those that couple via a hydroxy group (e.g. carbonyldiimidazole, butanediol diglycidyl ether, dialdehydes, diisothiocyanates). Polymers may also be formed by simple condensation chemistries to generate polymeric amino acids with the appropriate pKa (e.g. Gly-Gly).

In one embodiment, such immobilization, attachment and/or polymerization of the chemical species containing the ionizable group does not affect the pKa of the ionizable group, or leaves it in the desired ranges given above. In another embodiment, the chemical species is not coupled or polymerized via a positively ionizable nitrogen atom (for example, in contrast to the method described in W097/2982). In one aspect, the chemical species are immobilized, attached and/or polymerized via a hydroxyl group.

One polymeric material suitable for use in this method is a dimer or oligomer of Bis-Tris or Tris, or a material formed by attaching a plurality of Bis-Tris or Tris molecules to a polyacrylic acid backbone, e.g. by reacting Bis-Tris or Tris monomer with polyacrylic acid using 1-ethyl-3-dimethylaminopropyl carbodiimide (EDC). The polymer can then be easily separated from the reactants using dialysis against a suitable reagent or water. The polyacrylic acid may have molecular weight of between about 500 and 5 million or more, or between about 100,000 and 500,000.

The nature of the resultant Bis-Tris or Tris/polyacrylic acid molecule will depend on, the ratio of the coupled components, since the polymer will have different properties depending on the proportion of the acrylic acid groups that are modified with Bis-Tris or Tris. For example, carboxy groups may remain unmodified, as the presence of these will not prevent the Bis-Tris or Tris from binding nucleic acid at low pH (especially if the Bis-Tris or Tris is in excess), but their negative charge at higher pHs will assist with release of the nucleic acid. The molar ratio of Bis-Tris or Tris:carboxy groups (before attachment) may be between about 5:1 and 1:5, 3:1 and 1:3, 2:1 and 1:2, 1.5:1 and 1:1.5 or about 1:1.

The presence of high residual charge (i.e. charged species present in solution along with the extracted nucleic acid) may adversely affect the analysis of nucleic acids by PCR, or interfere with the binding of primers, dNTPs or polymerase to the nucleic acid, or to the sequestration of Mg²⁺ ions, which are essential to PCR. In one embodiment; residual positive charge is avoided.

In one embodiment, materials for use in the invention, such as the biological buffers described above, possess minimal residual positive charge (preferably minimal residual charge) at the pH at which the nucleic acid is released, and/or at pHs 8-8.5, making interference with or inhibition of downstream processes unlikely.

Further examples of charge switching molecules for nucleic acid purification are based on detergents or surfactants that have a hydrophobic portion and a hydrophilic portion which comprises a positively ionizable group with a suitable pKa, e.g. decyl methyl imidazole or dodecyl-Bis-Tris. These detergents/surfactants can be adsorbed onto surfaces e.g. plastic via their hydrophobic portions and the hydrophilic (ionizable) portions can be used to capture nucleic acid.

Another family of suitable materials for capture and easy release of nucleic acids are carbohydrates e.g. glucosamine, polyglucosamine (including chitosans), kanamycins and their derivatives, i.e. sugar ring based structures containing one or more nitrogen atoms surrounded by hydroxyl groups which may also contain other groups such as acetate or sulfate groups to provide a suitable pKa for binding and release of nucleic acids.

Another group of materials with suitable pKa values are nucleic acid bases, e.g. cytidine (pKa 4.2). These can be immobilized via hydroxy groups to a polymer or solid phase carboxy group using carbodiimides.

A still further group of materials having members with suitable pKa values are heterocyclic nitrogen-containing compounds. Such compounds may be aromatic or aliphatic and may be monomers, oligomers or polymers, such as morpholine-, pyrrole-, pyrrolidine-, pyridine-, pyridinol-, pyridone-, pyrroline-, pyrazole-, pyridazine-, pyrazine-, piperidone-, piperidine-, or piperazine-containing compounds, e.g. polyvinylpyridine. Such compounds may be substituted with electronegative groups to bring the pKa value(s) of the ionizable nitrogen atom(s) into an acceptable range, e.g. as defined above. However, in some compounds this may not be necessary, the pKa already being in such a range.

A still further group of charge switch materials for binding nucleic acid have surface amine groups, and in particular amine groups which are not polyamines. These monoamine groups can be represented by the formula —NR₁R₂, where R₁ and R₂ are hydrogen or substituted or unsubstituted alkyl. Although these materials typically have pKa values which at higher than those of materials used in preferred embodiments of the invention, they can be employed in the extracting of nucleic acid, optionally employing them with negatively charged species as described herein to modify the overall pKa of the charge switch material.

Further groups are materials that provide ionizable groups capable of acting as charge switch materials and binding nucleic acid are dyes, such as biological dyes having pKas between 5 and 8.

Some materials for use in the compositions and methods described herein are hydrophilic, for example those comprising charge switch materials which are (or which comprise chemical species which before immobilization or polymerization are) water soluble.

Once a suitable charge switch material has been prepared, repeated capture and release of nucleic acids can be performed by adjusting the pH up or down. Thus sequential reactions or analysis can be performed on the nucleic acids using the same charge switch material. For example, DNA can be isolated from a biological sample using a PCR tube comprising a charge switch material. Then, following PCR, the amplified DNA product may be isolated from the buffer constituents or primers by adjusting the pH in the same tube.

### EXAMPLES

Protocol

4 ml samples of reagents were prepared and added to 1 ml samples of blood added to a 15 ml tube. The tubes were mixed for 20 seconds and poured into a tray. Any flocculation was assessed by gently swirling the tray. The DNA nuclear precipitate was separated and dissolved in 1% SDS with Proteinase K (200·g/ml) overnight. A portion was subjected to electrophoresis on a 1% agarose gel to determine DNA content.

### Example 1

### Candidate Substances for Causing DNA Flocculation

After observing the flocculating effect of sodium chloride, a range of salts and other materials were tested in an elution buffer containing 10 mM Tris HCl at pH 8.5 at concentration ranges from 0 to 1.0 M.

Sodium chloride (NaCl) and ammonium bicarbonate (NH₄HCO₃) produced weak precipitation in a 0.1 M solution, increasing to medium levels at 0.25 M and strong precipitation at 0.5 M and above. Sodium hydrogen phosphate (NaHPO₄) produced weak flocculation between 0.5 and 1.0 M. Denaturants such as guanidine HCl and urea failed to produce any significant precipitation across the range tested. Calcium chloride (CaCl₂) also failed to produce any precipitation. Iron (III) chloride (FeCl₃) produced excessive levels of protein precipitation. Sodium hydroxide (NaOH) produced a viscous jelly and no observable flocculation. Potassium acetate/potassium chloride at pH 4.0 produced weak precipitation at 0.125 M and above.

Thus, the precipitation/flocculation reaction appeared to be produced by a solution of a monovalent cationic salt. The extent and rate of the flocculation produced was a function of the concentration of the monovalent cationic salt.

### Example 2

### Inclusion of Further Reagents

A series of further reagents were added to a solution of 0.5 M NaCl in elution buffer at pH 8.5 to determine their effect on the flocculation reaction. The further reagents were tested at concentrations of 0.1%, 1% and 10%. Some reagents were tested in the absence of the NaCl to show that the reaction could still take place.

Triton X-100, a non-ionic detergent, produced strong flocculation when added to the 0.5 M NaCl elution buffer. The strong ionic detergents sodium dodecyl sulfate (SDS) and lauryl sarcosine produced unusable jellies at all of the tested concentrations when added to the 0.5 M NaCl elution buffer. Polyethylene glycol 3500 (PEG3500), when added to elution buffer without NaCl, produced either very low levels of flocculation at 0.1% and 1% or gross levels of protein flocculation when added at 10%. Propanediol added to elution buffer without NaCl produced very low levels of flocculation.

### Example 3

### Varying the pH of the Elution Buffer

The pH of the elution buffer used in examples 1 and 2 was changed to pH 4 to see whether there would be any effect of the flocculation produced. The elution buffer contained 0.5 NaCl, to which was added 0, 0.1%, 1% and 10% Triton X-100, PEG3500 and propanediol.

As expected, the samples to which PEG3500 was added showed gross protein precipitation, while those with Triton X-100 produced flocculation suitable for use in separating the nuclear material and DNA from other cell components.

### Example 4

### Purification of RNA from HeLa Cells

CST magnetic beads were prepared by mixing carboxylated magnetic beads in a 10 fold excess of Bis-Tris in 0.1 M imidazole buffer, pH 6 with EDC at 10 mg/ml.

About 10⁶ cultured HeLa cells were suspended in 100 μl of PBS and added, dropwise, to a solution of 0.5 M NaCl containing 1% Triton X 100, 10 mM dithiothreitol (DTT) and 10 μl of CST magnetic beads. The tubes were mixed for 30 seconds or until aggregation was observed. The aggregate of beads containing DNA was separated and the supernatant was removed to a fresh tube. RNA was then isolated from the supernatant by precipitation of the RNA with isopropanol or ethanol followed by centrifugation.

### Example 5

### Purification of RNA

Supernatant containing RNA was separated from the DNA as described above. RNA in the supernatant was then bound by CST magnetic beads (also as above) by adjusting the supernatant to pH 4.5 with acetate buffer. The RNA was recovered in 10 mM Tris HCL pH 9.0, 1 mM EDTA.

### Example 6

### Purification of RNA

Supernatant containing RNA was separated from the DNA as described above. The supernatant was adjusted by adding Urea and LiCl to a final concentration of 3.5 M and 0.2 M respectively with 20 ug/ml Proteinase K and 20 mM DTT. Then 40ul of the same CST beads as described above were added in an acetate buffer at pH 4.5 to bind the RNA. The beads were washed and then the RNA eluted in 50 ul of 10 mM Tris HCl pH 9.0, 1 mM EDTA.

### Example 7

### Purification of RNA from Mouse Liver

About 10 mg of mouse liver was thoroughly homogenized in a solution containing 1% Triton X-100 and 0.5 M NaCl, LiCl, KCl or CsCl along with 10 μl of CST magnetic beads. The tubes were mixed for 30 seconds or until aggregation was observed. The aggregate of beads containing DNA was separated and the supernatant was removed to a fresh tube. Alternatively, the magnetic beads were omitted and the aggregated DNA was removed by centrifugation. RNA was then isolated from the supernatant by: 1) precipitation of the RNA with isopropanol or ethanol followed by centrifugation; or 2) binding the RNA using CST magnetic beads by adjusting the supernatant to pH 4.5 with an acetate buffer as described above. The RNA was recovered in 10 mM Tris-HCl, pH 9.0, 1 mM EDTA.

A β-actin PCR was then carried out on 0.5 μg of the isolated RNA against a DNA standard curve to determine the level of genomic DNA (gDNA) contamination. The resulting PCR products were quantified using the 2100 Bioanalyzer DNA 1000 chip assay (Agilent). The results are shown in Table 1.

TABLE 1

| Sample | PCR product (μg/ml) | Actual % gDNA |
|---|---|---|
| 10% control | 4.51 | 10 |
| 5% control | 2.23 | 5 |
| 2% control | 0.90 | 2 |
| 1% control | 0.70 | 1 |
| NaCl | 1.38 | 2.9 |
| LiCl | 1.02 | 2.0 |
| KCl | 0.95 | 1.9 |
| CsCl | 0.74 | 1.4 |

### Example 8

### Purification of RNA from Mouse Brain and Liver

About 10 mg of mouse brain and mouse liver was thoroughly homogenized in a solution containing 1% Triton X-100 and 0.5 M CsCl, either with or without 10 mM NaOH, along with 10 μl of CST magnetic beads. The tubes were mixed for 30 seconds or until aggregation was observed. The aggregate of beads containing DNA was separated and the supernatant was removed to a fresh tube. Alternatively, the magnetic beads were omitted and the aggregated DNA was removed by centrifugation. RNA was then isolated from the supernatant as described in Example 7.

A β-actin PCR was then carried out on 0.5 μg of the isolated RNA against a DNA standard curve to determine the level of genomic DNA (gDNA) contamination. The resulting PCR products were quantified using the 2100 Bioanalyzer DNA 1000 chip assay (Agilent). The results are shown in Table 2.

TABLE 2

| Sample | PCR product (μg/ml) | Actual % gDNA |
|---|---|---|
| 10% control | 9.82 | 10 |
| 5% control | 7.77 | 5 |
| 2% control | 5.36 | 2 |
| 1% control | 4.34 | 1 |
| Brain −NaOH | 3.79 | ≈0 |
| Brain +NaOH | 6.84 | 4.5 |
| Liver −NaOH | 4.10 | 0.1 |
| Liver +NaOH | 8.37 | 7.0 |

### Example 9

### Purification of RNA from HeLa Cells

About 1×10⁶ cultured HeLa cells were suspended in 100 μl of PBS and added, dropwise, to a solution containing 1% Triton X-100 and either 0.5 M NaCl or CsCl along with 10 μl of CST magnetic beads. The tubes were mixed for 30 seconds or until aggregation was observed. The aggregate of beads containing DNA was separated and the supernatant was removed to a fresh tube. Alternatively, the magnetic beads were omitted and the aggregated DNA was removed by centrifugation. RNA was then isolated from the supernatant as described in Example 7.

A β-actin real time PCR (qPCR) was then carried out on 0.5 μg of the isolated RNA using a real time PCR SYBRgreen assay against a DNA standard curve to determine the levels of gDNA contamination. The results are shown in Table 3.

TABLE 3

| Sample | C(T) | ng DNA in 500 ng RNA | Actual % gDNA |
|---|---|---|---|
| NaCl | 29.80 | 1.65 | 0.33 |
| CsCl | 31.19 | 0.75 | 0.15 |

### Example 10

### DNA Extraction from Blood

150 μl of well mixed, resuspended magnetic beads were placed into a 50 ml tube and 30 ml of lysis buffer was added. The magnetic beads were homogeneously distributed throughout the buffer, by swirling the tube gently. To this, 10 ml of a well mixed blood sample was added into the lysis Buffer/bead suspension, the tube capped and gently mixed by inverting three times. The tube was then incubated at RT for 5 minutes with periodic gentle inversion mixing. The tube was placed on a 50 ml tube magnetic separator and left for 3 minutes, after which the supernatant was removed using a 5 ml pipette without disturbing the bead pellet.

The tube was removed from the magnet, and 5 ml of fresh lysis buffer was added. The tube was re-capped, gently mixed by repeat inversion for about 10 seconds, then placed back on the magnet for about 20 seconds. The supernatant was then completely removed as described above. The tube was taken off the magnet, and 5 ml of digestion buffer and 40 μl of protease buffer were added. The capped tube was gently vortexed until the bead/pellet had been fully dispersed (about 20 seconds). The tube was then incubated at 65° C. in a water bath for 10 min. The digest was allowed to cool fully to room temperature, then 5 ml of 100% IPA was added.

The tube was gently rocked backwards and forwards until a visible aggregate had formed within the tube, leaving behind a clear, green colored supernatant. The tube was replaced on the magnetic separator and left for 30 seconds, after which the supernatant was removed using a 5 ml pipette. The tube was removed from the magnet, and 3 ml of 50% aqueous IPA wash was added and the tube gently rocked backwards and forwards for between 10-15 seconds, then placed back on the magnet. This was left for 20 seconds then the wash was completely removed with a 5 ml pipette. This was left for 1 min to allow any IPA Wash to drain to the bottom of the tube and was then completely removed with a 1 ml pipette. 0.25 ml of aqueous wash was gently pipetted over the DNA/bead pellet, left for 1 minute to allow it to fully drain to the bottom of the tube then completely removed with a 1 ml pipette. This was repeated with a further 0.25 ml of aqueous wash. All visible signs of liquid were removed from the bottom of the tube. The tube was taken off the magnet, 1 ml of elution buffer was added and the tube was gently swirled, ensuring that the whole DNA/bead pellet was released from the side of the tube and entered the elution buffer. The tube was incubated at 65° C. for 1 hour, followed by a very gentle tip—mix with a 1 ml pipette until the bead pellet had been completely re-dispersed. The tube was placed on the magnetic separator and left for about 15 min. Without disturbing the bead pellet, the Elution Buffer containing the genomic DNA was removed using a 1 ml pipette and placed into a clean 2 ml tube.

The references mentioned herein are all expressly incorporated by reference in their entirety.

We claim:

1. A method for separating genomic DNA from a cell sample, comprising:
(a) lysing said cell sample under non-denaturing conditions to flocculate said genomic DNA and form a precipitate; and
(b) collecting said precipitate.

2. The method of claim 1, wherein said cells are lysed with a hypertonic monovalent cationic salt solution at a pH between about 4.0 and 10.0.

3. The method of claim 1, wherein said cell lysis occurs in the presence of a solid phase capable of binding said genomic DNA.

4. The method of claim 1, wherein said lysing does not comprise using one or more of:
(i) a chaotropic reagent;
(ii) a strong ionic detergent;
(iii) a pH that is above about 10.0 or below about 4.0;
(iv) a divalent or trivalent metal ion; or
(v) a protein precipitant.

5. The method of claim 1, wherein said method does not involve ultracentrifugation.

6. The method of claim 2, wherein said salt is an alkali metal cationic salt or an ammonium salt.

7. The method of claim 6, wherein said salt is sodium chloride (NaCl), potassium chloride (KCl), sodium carbonate (Na₂CO₃), sodium bicarbonate (NaHCO₃), ammonium bicarbonate (NH₄HCO₃), lithium chloride (LiCl) or Cesium Chloride (CsCl).

8. The method of claim 2, wherein the concentration of said hypertonic monovalent cationic salt solution is between about 10 mM and 1.0 M.

9. The method of claim 2, wherein said hypertonic monovalent cationic salt solution has a pH of between about 6.0 and 9.0.

10. The method of claim 2, wherein said hypertonic monovalent cationic salt solution further comprises about 0.1 to 1.0% v/v of a non-ionic detergent.

11. The method of claim 1, wherein said cell sample is a mammalian cell sample or a blood cell sample.

12. The method of claim 11, wherein said cell sample is a whole blood cell sample.

13. The method of claim 3, wherein said solid phase binds at least about 50·g of genomic DNA per mg of solid phase.

14. The method of claim 3, wherein said solid phase binds at least about 100·g of genomic DNA per mg of solid phase.

15. The method of claim 3, further comprising:
(a) contacting said solid phase with a solution under conditions to release said precipitate of said genomic DNA and nuclear material into said solution;
(b) treating said solution to remove one or more impurities; and
(c) rebinding said genomic DNA to said solid phase.

16. The method of claim 15, further comprising contacting said solution in (b) with a protease.

17. The method of claim 15, wherein said rebinding in (c) comprises adding a precipitant, whereby said genomic DNA rebinds to said solid phase.

18. The method of claim 3, wherein said solid phase is a charge switch material.

19. The method of claim 3, wherein said solid phase is a spooling rod, a bead or particulate composition, a single bead, a mesh, a membrane, a sinter, a plastic support, a paper, a tip, a dipstick, a wall of a container, a tube, a well, a probe, a pipette, a filter, a sheet, a slide or a plug.

20. The method of claim 1, further comprising purifying said precipitate or amplifying a nucleic acid sequence within said genomic DNA.

21. A method for separating RNA from a cell sample, the method comprising:
(a) lysing said cells under non-denaturing conditions to flocculate genomic DNA and form a precipitate and a supernatant; and
(b) collecting said supernatant from said precipitate, wherein said supernatant contains said RNA.

22. The method of claim 21, wherein said cells are lysed with a hypertonic monovalent cationic salt solution at a pH between about 4.0 and 10.0.

23. The method of claim 21, wherein said cell lysis occurs in the presence of a solid phase capable of binding said genomic DNA.

24. The method of claim 21, wherein said cell lysis does not comprise using one or more of:
(i) a chaotropic reagent; or
(ii) a strong ionic detergent; or
(iii) a pH that is above 10.0 or below 4.0; or
(iv) a divalent or trivalent metal ion; or
(v) a protein precipitant.

25. The method of claim 21, wherein at least about 90% of the protein initially present in said cell sample is removed.

26. The method of claim 22, wherein said salt is an alkali metal cationic salt or an ammonium salt.

27. The method of claim 26, wherein said salt is sodium chloride (NaCl), potassium chloride (KCl), sodium carbonate (Na₂CO₃), sodium bicarbonate (NaHCO₃), ammonium bicarbonate (NH₄HCO₃), lithium chloride (LiCl) or Cesium Chloride (CsCl).

28. The method of claim 22, wherein the concentration of said hypertonic monovalent cationic salt solution is between about 10 mM and 1.0 M.

29. The method of claim 22, wherein said hypertonic monovalent cationic salt solution has a pH between about 6.0 and 9.0.

30. The method of claim 22, wherein said hypertonic monovalent cationic salt solution further comprises about 0.1 to 1.0% v/v of a non-ionic detergent.

31. The method of claim 21, wherein the cell sample is a mammalian cell sample or a blood cell sample.

32. The method of claim 31, wherein said cell sample is a whole blood cell sample.

33. The method of claim 23, wherein said solid phase binds at least 50 μg of genomic DNA per mg of solid phase.

34. The method of claim 23, wherein said solid phase binds at least 100 μg of genomic DNA per mg of solid phase.

35. The method of claim 23,further comprising:
(a) contacting said solid phase with a solution under conditions to release the precipitate of said genomic DNA and nuclear material into said solution;
(b) treating said solution to remove one or more impurities; and
(c) rebinding said genomic DNA to said solid phase.

36. The method of claim 35, further comprising contacting said solution in (b) with a protease.

37. The method of claim 35, wherein said rebinding in (c) comprises adding a precipitant, whereby said genomic DNA rebinds to said solid phase.

38. The method of claim 23, wherein said solid phase is a charge switch material.

39. The method of claim 23, wherein the solid phase is a spooling rod, a bead or particulate composition, a single bead, a mesh, a membrane, a sinter, a plastic support, a paper, a tip, a dipstick, a wall of a container, a tube, a well, a probe, a pipette, a filter, a sheet, a slide or a plug.

40. The method of claim 21, further comprising purifying said RNA.
